(19) United States
(12) Patent Application Publication
(10) Pub. No.: US 2011/0190613 A1
(43) Pub. Date: Aug. 4, 2011

(54) HYBRID SPECTROPHOTOMETRIC MONITORING OF BIOLOGICAL CONSTITUENTS

(75) Inventors: Wei ZHANG, San Jose, CA (US); Zengpin Yu, Palo Alto, CA (US); Shih-Ping Wang, Los Altos, CA (US)

(73) Assignee: O2 MEDTECH, INC.,, Los Altos, CA (US)

(21) Appl. No.: 13/004,393

(22) Filed: Jan. 11, 2011

Related U.S. Application Data
(60) Provisional application No. 61/293,805, filed on Jan. 11, 2010.
(60) Provisional application No. 61/298,890, filed on Jan. 27, 2010.
(60) Provisional application No. 61/312,673, filed on Mar. 11, 2010.

(51) Int. Cl. A61B 5/1455

(52) U.S. Cl. 600/328

(57) ABSTRACT

Systems, methods, and related computer program products for non-invasive NIR spectrophotometric (NIRS) monitoring of total blood hemoglobin levels and/or other blood constituent levels based on a hybrid combination of phase modulation spectrophotometry (PMS) and continuous wave spectrophotometry (CWS) are described. PMS-based measurements including both amplitude and phase information used in the determination of a non-pulsatile component of an absorption property for each of at least three distinct wavelengths are processed to compute PMS-derived intermediate information at least partially representative of a scattering characteristic. CWS-based measurements including amplitude information is processed in conjunction with the PMS-derived intermediate information to compute a pulsatile component of the absorption property. A metric representative of at least one chromophore level, such as the total blood hemoglobin level, is computed from the pulsatile component of the absorption property at the at least three wavelengths and displayed on an output display.

### CROSS-REFERENCE TO RELATED APPLICATIONS

This patent application claims the benefit of the following provisional patent applications, each of which is incorporated by reference herein: U.S. Ser. No. 61/293,805, filed Jan. 11, 2010; U.S. Ser. No. 61/298,890, filed Jan. 27, 2010; and U.S. Ser. No. 61/312,673, filed Mar. 11, 2010. The subject matter of this patent application is related to the subject matter of the following patent applications, each of which is incorporated by reference herein: U.S. Ser. No. 12/701,274 filed Feb. 5, 2010 (Atty. Dkt. 6949/81341); U.S. Ser. No. 12/815,696, filed Jun. 15, 2010 (Atty. Dkt. 6949/81719); U.S. Ser. No. 12/826,218, filed Jun. 29, 2010 (Atty. Dkt. 6949/81720); and U.S. Ser. No. 12/832,603, filed Jul. 8, 2010 (Atty. Dkt. 6949/81721).

### FIELD

This patent specification relates to the non-invasive monitoring of a physiological condition of a patient using information from near-infrared (NIR) optical scans. More particularly, this patent specification relates to systems, methods, and related computer program products for non-invasive NIR spectrophotometric (NIRS) monitoring of total hemoglobin levels and/or other blood constituent levels.

### BACKGROUND AND SUMMARY

Hemoglobin is an iron-containing metalloprotein contained in red blood cells that serves as a basis for oxygen transport from the lungs to the various tissues of the body. Hemoglobin exists in the body in both oxygenated and deoxygenated states, the total hemoglobin (HbT) level being equal to the sum of the oxygenated hemoglobin (HbO) level and the deoxygenated hemoglobin (Hb) level for any particular biological volume or compartment. Hemoglobin levels are most often expressed as concentrations in grams per liter deciliter (g/dl). As used herein, total hemoglobin concentration is denoted as [HbT], oxygenated hemoglobin concentration is denoted as [HbO], and deoxygenated hemoglobin is denoted as [Hb].

The use of near-infrared (NIR) light as a basis for the measurement of biological properties or conditions in living tissue is particularly appealing because of its relative safety as compared, for example, to the use of ionizing radiation. Various techniques have been proposed for non-invasive NIR spectroscopy or NIR spectrophotometry (NIRS) of biological tissue. The following commonly assigned patent applications, each of which is incorporated by reference herein, are generally directed to the continuous, non-invasive, real-time NIR spectrophotometric detection of an oxygen saturation metric [SO₂], which refers to the fraction or percentage of total hemoglobin [HbT] that is oxygenated hemoglobin: U.S. Ser. No. 12/701, 274, supra; U.S. Ser. No. 12/815,696, supra; U.S. Ser. No. 12/826,218, supra; and U.S. Ser. No. 12/832,603, supra.

Although [SO₂] readings provide valuable insight into the patient's condition, especially when localized to the brain tissue, another highly useful metric for monitoring and/or evaluating the condition of the patient is the total hemoglobin concentration [HbT] itself, as measured in grams per deciliter of the biological volume or compartment under study. In traditional clinical practice, the total hemoglobin [HbT] is measured using an invasive blood draw, and then testing the drawn blood sample in a hospital laboratory using a CO-oximeter or other laboratory equipment. Point-of-care devices based on spectrophotometry or electrical conductivity testing of smaller blood samples obtained by finger prick have also been introduced, wherein the results can be obtained more quickly, but these devices are still invasive in nature and of lesser established accuracies compared to the CO-oximeter “gold standard.” It would be desirable to provide for continuous, real-time, non-invasive monitoring of total hemoglobin [HbT] in a convenient, efficient, and accurate manner. Among other clinical benefits, such a system would be highly advantageous in a surgery environment, where continuous [HbT] monitoring could facilitate the avoidance of unnecessary blood transfusions, facilitate cost decreases by more effective titration of blood, and/or facilitate the initiation of more time blood transfusions, when appropriate. Such system could further streamline emergency room practice, for example, by facilitating quick identification of chronic or acute anemia conditions, increasing efficiencies through rapid testing and triage. In critical care environments, hemorrhaging could be identified earlier, thereby increasing patient safety by allowing for more timely intervention. Other issues arise as would be apparent to a person skilled in the art in view of the present disclosure.

One or more preferred embodiments described further hereinbelow are directed to the non-invasive NIRS-based monitoring of total hemoglobin [HbT] levels, and/or other biological constituents contained in the blood of a patient, based on the monitoring of pulsatile variations (i.e., variations occurring at a rate of the patient's heartbeat, usually in the range of 0.5 Hz-4 Hz) in one or more NIRS-based measurements as discriminated from longer term, non-pulsatile components thereof of the NIRS-based measurements. Phase modulation spectrophotometry (PMS) systems, which are sometimes termed intensity modulation spectroscopy systems and sometimes termed frequency domain spectroscopy systems, are known in the art and are discussed, for example, in U.S. Pat. No. 4,972,331, U.S. Pat. No. 5,187,672, and WO1994/21173A1, each of which is incorporated by reference herein. Generally speaking, PMS-based NIRS systems are characterized by a relatively high modulation rate, usually in the range of 100 MHz-1000 MHz, and are further characterized in that both intensity measurements and phase measurements for the detected radiation are processed to compute a characteristic of the biological volume being monitored. Continuous wave spectrophotometry (CWS) systems are also known in the art and are discussed, for example, in W01992/20273A2 and WO1996/16592A1, each of which is incorporated by reference herein. Generally speaking, CWS-based NIRS systems are characterized by a relatively low modulation rate, usually well below 1 MHz and typically only around 25 kHz or lower, not tending all the way to DC primarily to avoid unacceptable 1/f noise levels, and are further characterized in that intensity measurements are processed to compute a characteristic of the biological volume is used measurements without regard to any measured phase information.

As further discussed in the commonly assigned U.S. Ser. No. 12/701,274, supra, PMS-based NIRS systems offer certain advantages over CWS-based NIRS systems, while at the same time suffering from selected disadvantages not suffered by CWS-based NIRS systems. On the one hand, PMS-based measurements can be generally viewed as being more accurate and precise than CWS-based measurements in that both the absorption and scattering properties of the biological volume can be computed from the measured amplitude and phase information. In contrast, for CWS-based measurements, it is required to that a pre-existing estimate of a scattering property or a scattering-related characteristic of the biological volume be used, with the absorption property of the biological volume then being computed from the measured amplitude information in conjunction with that pre-existing estimate. As illustrated in FIG. 16, the scattering property can vary from patient to patient and over time, and therefore the use of such a pre-existing estimate can lead to inaccurate results. can be different for different patients, and can On the other hand, PMS-based systems contain certain practical limitations compared to CWS-based systems, including the need for substantially more complex and expensive modulation and demodulation circuitry, a more limited penetration depth, and higher sensitivity to noise and ambient electromagnetic interference. In comparison to CWS-based systems, it is particularly difficult and expensive to realize PMS-based systems that are capable of measurement rates sufficiently high to accurately detect pulsatile variations in the measured absorption and scattering properties.

For one or more preferred embodiments, it has been found particularly advantageous to combine certain aspects of PMS-based monitoring with certain aspects of CWS-based monitoring to result in an overall “hybrid” system that exhibits key advantages associated with the different spectrophotometric strategies, while not exhibiting certain disadvantages suffered when each strategy is used individually. Although a hybrid combination of PMS-based and CWS-based monitoring has been found to be advantageous, it is to be appreciated that the scope of the present teachings is not so limited, and that hybrid combinations of PMS-based monitoring with one or more non-PMS-based monitoring types other than CWS-based monitoring is also within the scope of the present teachings.

Provided according to one preferred embodiment is a method for near-infrared spectrophotometric (NIRS) monitoring of at least one chromophore level in a biological volume of a patient, comprising determining a non-pulsatile component of an absorption property of the biological volume for each of at least three distinct wavelengths of near-infrared radiation using a phase modulation spectrophotometry (PMS) based measurement method. The PMS-based measurement method is characterized by a relatively high modulation rate and is further characterized in that both amplitude and phase information detected at the relatively high modulation rate are processed to compute the non-pulsatile component of the absorption property. The method further comprises processing the measured amplitude and the measured phase information associated with the PMS-based determination of the non-pulsatile component of the absorption property to compute PMS-derived intermediate information that is at least partially representative of a scattering characteristic of the biological volume. The method further comprises determining a pulsatile component of the absorption property of the biological volume for each of the at least three distinct wavelengths using a continuous wave spectrophotometry (CWS) based measurement method characterized by a relatively low modulation rate. Amplitude information detected at the relatively low modulation rate is processed in conjunction with the PMS-derived intermediate information to compute the pulsatile component of the absorption property. The method further comprises computing at least one metric representative of the at least one chromophore level in the biological volume based on the pulsatile component of the absorption property at the at least three wavelengths, and displaying the at least one metric on an output display.

Also provided is an apparatus for non-invasive NIRS monitoring of at least one chromophore level in a biological volume of a patient, comprising a probe assembly including a plurality of source-detector pairs configured to introduce near-infrared radiation into the biological volume and receive near-infrared radiation from the biological volume, and a processing and control device coupled to the plurality of source-detector pairs of the probe assembly. The processing and control device is configured to operate at least one of the source-detector pairs in a PMS mode, the PMS mode being characterized by a relatively high modulation rate and being further characterized in that both amplitude and phase information are detected and processed to determine an absorption property. The processing and control device is further configured to operate at least one of the source-detector pairs in a CWS mode, the CWS mode being characterized by a relatively low modulation rate and being further characterized in that amplitude information is detected and processed to determine the absorption property without regard to phase information. The apparatus further comprises an output display coupled to the processing and control device. A non-pulsatile component of an absorption property of the biological volume is determined for each of at least three distinct wavelengths based on measurements acquired in the PMS mode. The measurements acquired in the PMS mode are processed to compute PMS-derived intermediate information that is at least partially representative of a scattering characteristic of the biological volume. A pulsatile component of the absorption property of the biological volume is determined for each of the at least three distinct wavelengths based on measurements acquired in the CWS mode, wherein the determination includes processing the CWS-mode measurements in conjunction with the PMS-derived intermediate information to compute the pulsatile component of the absorption property. At least one metric representative of the at least one chromophore level in the biological volume is computed based on the pulsatile component of the absorption property at the at least three wavelengths, and the at least one metric is displayed on the output display.

Also provided is a method for providing an improved apparatus for NIRS monitoring of at least one chromophore level in a biological volume of a patient based on a pre-existing NIRS monitoring apparatus. The pre-existing NIRS monitoring apparatus includes a probe assembly, a processing and control device, and an output display. The pre-existing NIRS monitoring apparatus is operable in a pre-existing CWS mode characterized in that (i) a relatively low modulation rate is used, (ii) amplitude information is detected and processed according to a pre-existing algorithm to determine an absorption property without regard to phase information, and (iii) the pre-existing algorithm incorporates a pre-existing estimate of a scatter-related characteristic of the biological volume in the determination of a pulsatile absorption property, the pre-existing NIRS monitoring apparatus computing the at least one chromophore level based on the pulsatile absorption property and displaying the at least one chromophore level on the output display. The probe assembly and the processing and control device of the pre-existing NIRS monitoring apparatus are modified to be operable in a PMS mode in addition to the pre-existing CWS mode, the PMS mode being characterized by a relatively high modulation rate and being further characterized in that both amplitude and phase information are detected. The processing and control device is further modified to be operable to compute an actual version of the scatter-related characteristic for the biological volume based on measurements acquired in the PMS mode, and to incorporate the actual version of the scatter-related characteristic in place of the pre-existing estimate thereof in the pre-existing algorithm that determines the pulsatile absorption property. Advantageously, the modified version of the pre-existing NIRS monitoring apparatus provides improved monitoring of the at least one chromophore level by virtue of incorporating an actual, patient-specific, updated version of the scatter-related characteristic in place of the pre-existing estimate thereof in computing the at least one chromophore level.

### BRIEF DESCRIPTION OF THE DRAWINGS

FIG. 1A illustrates near-infrared spectrophotometric (NIRS) monitoring of a biological volume according to a preferred embodiment in which the biological volume is a head of a patient;

FIGS. 1B-1C illustrate examples of cerebral NIRS mounting probes according to one or more preferred embodiments;

FIG. 1D illustrates NIRS monitoring of a biological volume according to a preferred embodiment in which the biological volume is a fingertip of the patient;

FIGS. 1E-1F illustrate examples of fingertip NIRS mounting probes according to one or more preferred embodiments;

FIG. 1G illustrates NIRS monitoring of at least one chromophore level in a biological volume of a patient according to a preferred embodiment;

FIGS. 2A-2B illustrate a compartment model of a biological tissue volume that experiences pulsatile variations;

FIGS. 2C-2F illustrate mathematical expressions related to the computation of at least one metric representative of a biological constituent level in a biological volume based on computed pulsatile variations of an absorption property thereof at three distinct wavelengths according to a preferred embodiment;

FIGS. 3A-3B illustrate mathematical expressions related to the computation of a biological constituent level in a biological volume based on computed non-pulsatile variations of an absorption property thereof at three distinct wavelengths according to a preferred embodiment;

FIG. 4A and FIGS. 4B-5C illustrate a conceptual diagram and mathematical relationships, respectively, associated with a slope method for computing an absorption coefficient, a scattering coefficient, and an oxygen saturation metric for a biological volume;

FIG. 6 illustrates a switching method for introducing optical radiation introduction into a biological volume according to a preferred embodiment;

FIG. 7 illustrates a multiple frequency method for introducing optical radiation introduction into a biological volume according to a preferred embodiment;

FIGS. 8-10 illustrates NIRS monitoring of at least one chromophore level in a biological volume of a patient according to one or more preferred embodiments;

FIG. 11 illustrates demodulation of a detected optical signal to extract a pulsatile component thereof according to a preferred embodiment;

FIG. 12 illustrates mathematical relationships associated with the method of FIG. 8;

FIG. 13 illustrates a bilateral cerebral pulse oximetry system according to a preferred embodiment;

FIGS. 14-15 illustrate modifying an existing non-PMS-based NIRS measurement system, such as a CWS-based NIRS measurement system, according to a preferred embodiment;

FIG. 16 illustrates a plot of clinically measured values for a scattering property as measured on the forehead of a population of test patients;

FIG. 17 illustrates non-invasive NIRS monitoring of blood total hemoglobin concentration [HbT]_{A} according to a preferred embodiment; and

FIG. 18 illustrates a plot of a series of mappings between tissue total hemoglobin concentration and blood total hemoglobin concentration for use in the method of FIG. 17.

### DETAILED DESCRIPTION

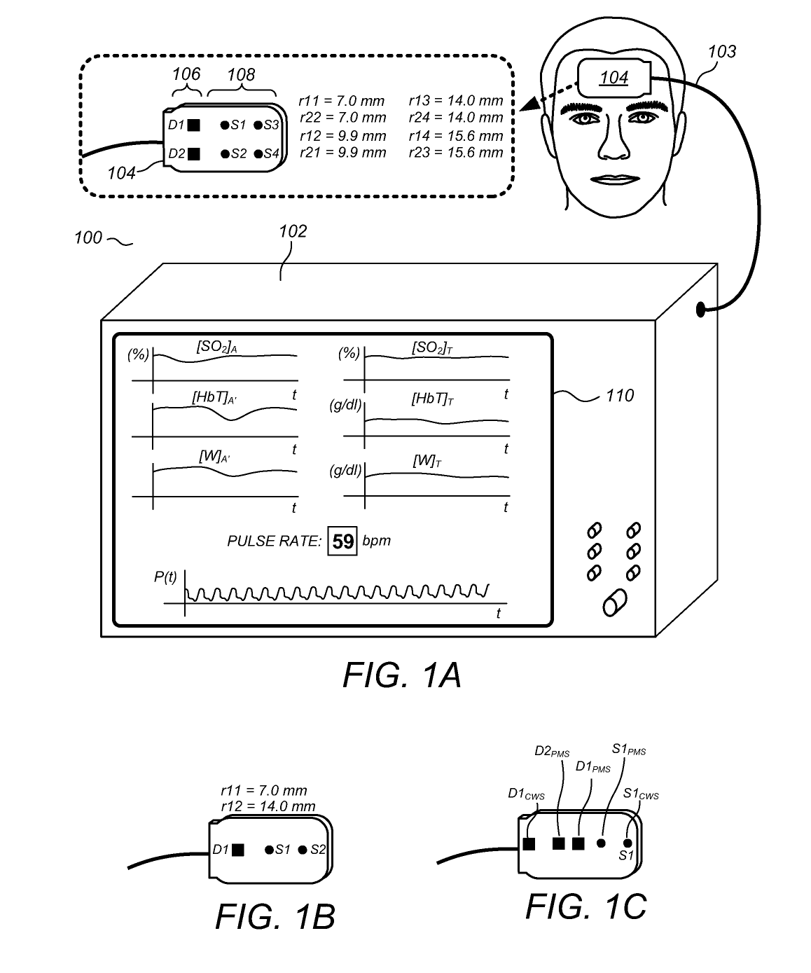

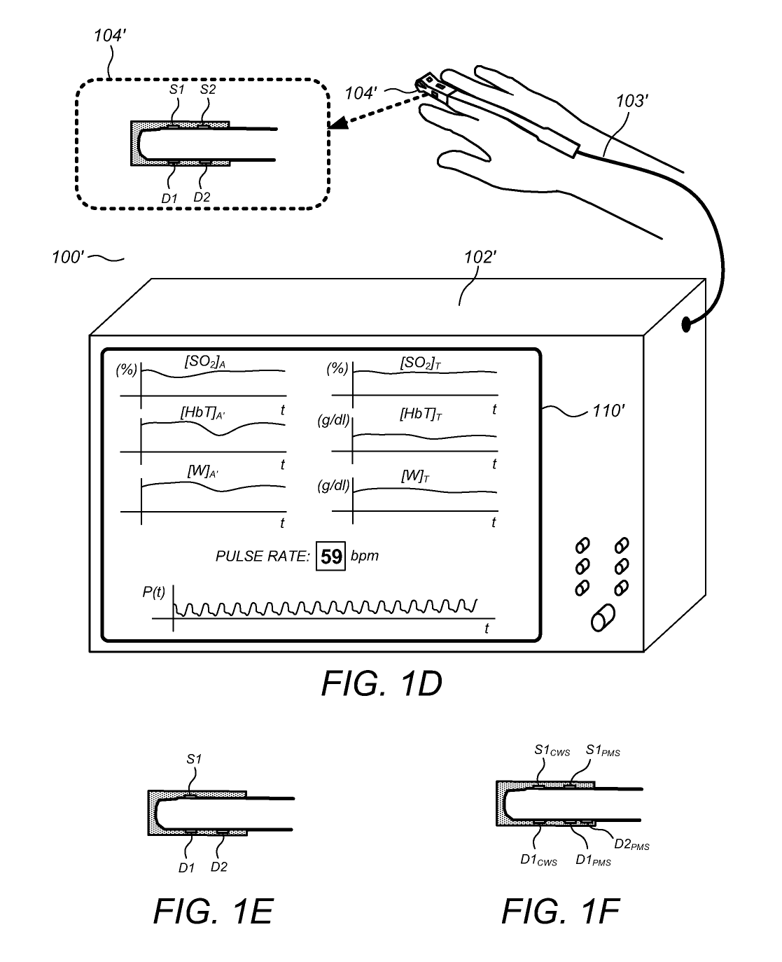

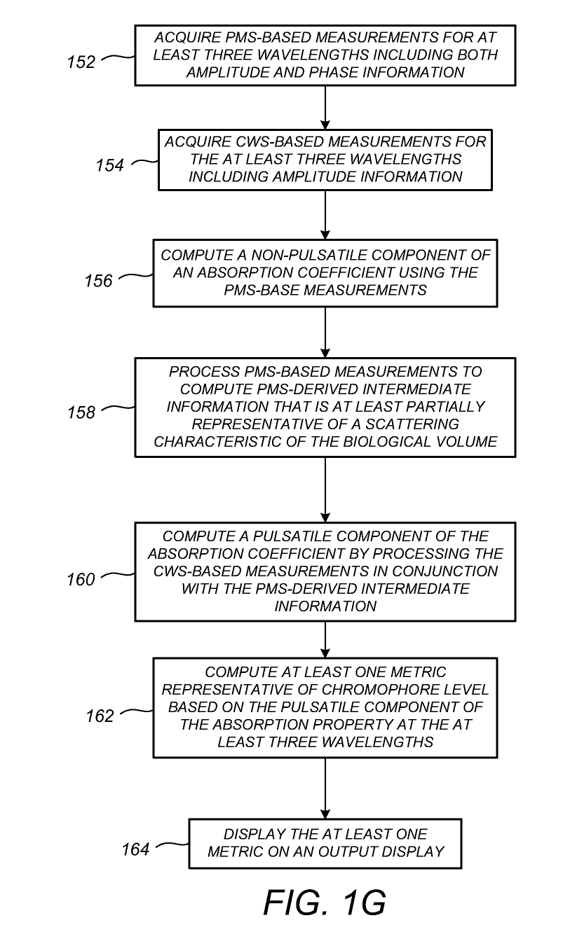

FIG. 1A illustrates an example of continuous, real-time, non-invasive total hemoglobin concentration [HbT] monitoring according to a preferred embodiment. Without loss of generality, for one preferred embodiment, the biological volume under study is modeled as consisting of a pulsatile (“arterial”) blood compartment “A” and a non-pulsatile (“tissue”) compartment “T”. The pulsatile compartment “A” and the tissue compartment “T” are modeled as each consisting of “M” chromophores. The number of chromophores M should be at least three, including a first chromophore that is oxygenated hemoglobin (HbO), a second chromophore that is deoxygenated hemoglobin (Hb), and a third chromophore that is water (W). As used herein, the symbols [HbO]_{A}, [Hb]_{A}, and [W]_{A} represent the respective concentrations of oxygenated hemoglobin, deoxygenated hemoglobin, and water in the arterial blood compartment, while the symbols [HbO]_{T}, [Hb]_{T}, and [W]_{T} represent the respective concentrations of oxygenated hemoglobin, deoxygenated hemoglobin, and water in the tissue compartment. The number of different wavelengths “N” used should be equal to or greater than the modeled number of chromophores “M”.

While the examples herein are presented in the context of a three-chromophore (M=3) model and a three-wavelength (N=3) spectrophotometric scheme for clarity of description, it is to be appreciated that the number of chromophores “M” (and therefore the number of wavelengths “N”) can be readily extended to greater numbers (for example, four, five, six, or seven, and perhaps even up to 32 or greater) without departing from the scope of the present teachings. Examples of additional chromophores that can be included in the model are carboxyhemoglobin (HbCO) and methemoglobin (HbMet).

Included in FIG. 1A is an NIR-based monitoring system 100 according to a preferred embodiment, including a console unit 102 coupled via a coupling cable 103 to an NIR probe patch 104. The hardware setup and methodologies for the NIR-based monitoring system 100 can generally be similar to those described in one or more of the commonly assigned U.S. Ser. No. 12/701, 274, supra, U.S. Ser. No. 12/815,696, supra, U.S. Ser. No. 12/826,218, supra, and U.S. Ser. No. 12/832,603, supra, with adaptations as described herein for measuring total hemoglobin [HbT] and other useful metrics, such adaptations including, for example, closer spacing of the source-detector pairs (see FIG. 1A, sources 108 and detectors 106), and the use of an additional third source wavelength of λ3=1050 nm in addition to the two source wavelengths λ1=690 nm and λ2=830 nm. It is to be appreciated that the use of the three wavelengths λ1=690 nm, λ2=830 nm, and λ3=1050 nm is by way of example only and not by way of limitation.

The NIR probe patch 104 is preferably positioned on the patient's body at a location where there is a higher population of capillaries and/or where there is better blood circulation. One particularly advantageous location for the NIR probe patch is the forehead, as shown in FIG. 1A. Possible motion of the body part where the NIR probe patch is placed could be another major source of noise, since the [HbT] signal can be relatively weak. Thus, selection of where to place the probe becomes important. The forehead, chest, or other areas of the body without too much fat are good locations for the NIR probe patch.

For the preferred embodiment of FIG. 1A, NIR probe patch 104 includes sources 108 and detectors 106 as shown that establish at least one “far” source-detector spacing and at least one “near” source-detector spacing, for accommodating a semi-infinite slope method for absorption and attenuation coefficient computation as detailed further in Ser. No. 12/826,218, supra (hereinafter “the '218 application”). For the preferred embodiment of FIG. 1A, there is a sufficient multiplicity of source-detector pairs so as to establish two (or more) “far” source-detector spacings and two (or more) “near” source-detector spacings, such that variations in skin coupling factors and/or source/detector efficiency factors can be reliably accommodated, as also described in the '218 application, supra. In other preferred embodiments there can be fewer source-detector pairs, such as shown in FIG. 1B in which there are two source-detector pairs (a single “near” pair and a single “far” pair such that the slope method can still be used, with coupling/efficiency factors being assumed constant or compensated in other ways).

FIG. 1C illustrates a probe assembly according to another preferred embodiment, including a first source-detector pair S1_{CWS}-D1_{CWS} that is dedicated for CWS functionality, and two additional source-detector pairs S1_{PMS}-D1_{PMS} and S1_{PMS}-D2_{PMS} (the source S1_{PMS} being in common to the two additional source-detector pairs) that are dedicated for PMS functionality. It is to be appreciated that a wide variety of different probe assembly configurations are within the scope of the present teachings, ranging from a complete segregation of PMS source-detector pairs from CWS source-detector pairs as in FIG. 1C (i.e., the PMS source-detector pairs perform no CWS functionality and the CWS source-detector pairs perform no PMS functionality) to a complete integration of PMS source-detector pairs with CWS source-detector pairs (i.e., the same source-detector pairs perform both CWS and PMS functionality), including a wide variety of combinations lying between these two extremes, such as using each optical source to transmit both PMS and CWS modulated signals, but using distinct optical detectors to perform the respective PMS and CWS signal detections. A similarly wide variety of different probe assembly configurations can also be used in conjunction with the finger-mounted probes of FIGS. 1E-1F infra, and other probes for neck, chest, abdomen, etc., all being within the scope of the present teachings.

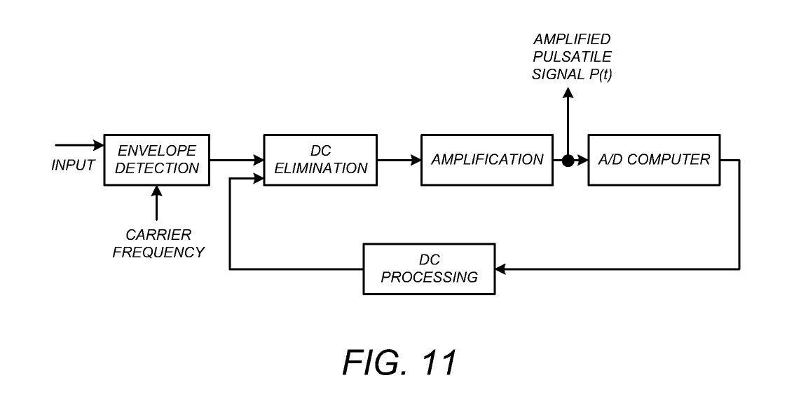

According to a preferred embodiment, based on methods for computing these quantities as disclosed herein, the NIR-based monitoring system 100 provides a real-time display 110 of an arterial hemoglobin saturation metric [SO₂]_{A}, a tissue hemoglobin saturation metric [SO₂]_{T} (i.e., applicable for the biological volume as a whole), an arterial hemoglobin concentration metric [HbT]_{A′}, a tissue hemoglobin concentration [HbT]_{T}, an arterial water concentration metric [W]_{A′}, and a tissue water concentration [W]_{T}. Also provided on the real-time display is a digital readout of the pulse rate of the patient, as well as a plot P(t) that serves as a pulse monitor waveform. The signal P(t) can be derived from a single detector signal intensity by controlled DC component removal and pulsatile component amplification as shown in FIG. 11, and/or can be derived from similar processing of multiple detector signal intensities and averaging methods.

FIG. 1D illustrates an NIR-based monitoring system 100′ according to a preferred embodiment, including a console unit 102′ coupled via a coupling cable 103′ to a finger-mounted probe 104′. It is to be appreciated that it would not be outside the scope of the preferred embodiments for an NIR probe patch to be provided that is positioned on the neck of the patient, or other location on the abdomen, arms, or legs. As with the probe patch 104 of FIG. 1A, the finger-mounted probe 104′ of FIG. 1D can be supplied with a full complement of source-detector pairs (e.g., enough to accommodate the “slope method” in a “balanced” configuration to cancel out skin coupling/efficiency variations), or with a fewer number of source-detector pairs, such as two source-detector pairs as in FIG. 1E. Shown in FIG. 1F is a finger-mounted probe assembly according to another preferred embodiment, including a first source-detector pair S1_{CWS}-D1_{CWS} that is dedicated for CWS functionality, and two additional source-detector pairs S1_{PMS}-D1_{PMS} and S1_{PMS}-D2_{PMS} (the source S1_{PMS} being in common to the two additional source-detector pairs) that are dedicated for PMS functionality. As with the cerebral monitoring probes of FIGS. 1A-1C, it is to be appreciated that a wide variety of different probe assembly configurations are within the scope of the present teachings for the finger-mounted probes of FIGS. 1D-1F, ranging from a complete segregation of PMS source-detector pairs from CWS source-detector pairs, to a complete integration of PMS source-detector pairs with CWS source-detector pairs, and a wide variety of combinations lying between these two extremes

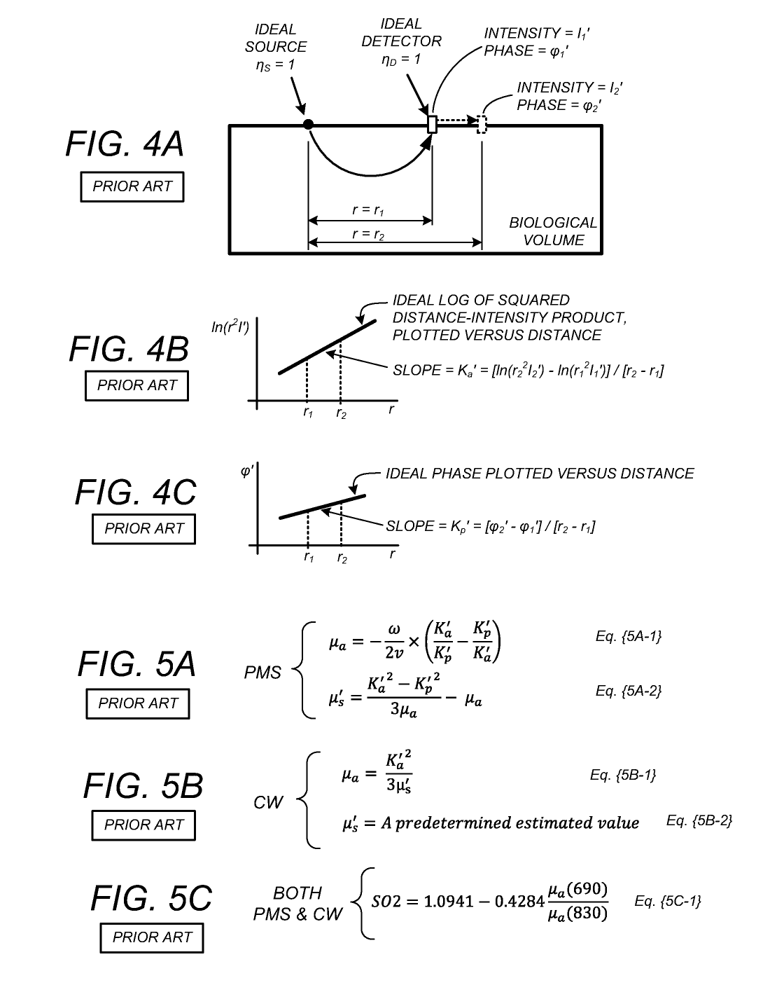

FIG. 1G illustrates NIRS monitoring of at least one chromophore level in a biological volume of a patient according to a preferred embodiment. At step 152, PMS-based measurements for each of at least three distinct wavelengths of near-infrared radiation are acquired. At step 154, CWS-based measurements for each of the at least three distinct wavelengths of near-infrared radiation are acquired. At step 156, a non-pulsatile component of an absorption property of the biological volume is computed from the PMS-based measurements for each wavelength. For one preferred embodiment, step 156 is carried out based on the slope method illustrated in FIGS. 4A-4C and FIG. 5A, with the resultant absorption coefficient μₐ being shown in Eq. {5A-1}. For most PMS implementations, the resultant absorption coefficient μₐ from step 156 will intrinsically be a non-pulsatile, since most PMS systems are not fast enough to keep up with the rate of the patient's heartbeat while also being economical, and an economical PMS system is preferred. However, the scope of the present teachings is not necessarily so limited, and in the event a known or hereinafter developed PMS measurement system can at least partially keep up with the heart rate of the patient, the resultant absorption coefficient μₐ can be low-pass filtered and provided in a non-pulsatile version.

At step 158, the PMS-based measurements are further processed to compute PMS-derived intermediate information that is at least partially representative of a scattering characteristic of the biological volume. One example of such PMS-derived intermediate information is a scattering coefficient μ′ₛ for each wavelength, which can be provided based on the relationship of Eq. {5A-2}, as is detailed further hereinbelow with respect to step 808 of FIG. 8. Another example of PMS-derived intermediate information is a differential pathlength factor (DPF) for each wavelength. The DPF can be computed at each respective wavelength from the absorption coefficient μₐ (non-pulsatile) and the scattering coefficient μ′ₛ using a known relationship, such as that shown in Eq. {1} (assuming an infinite medium, as might be assumed for the finger) and Eq. {2} (assuming a semi-infinite medium, as might be assumed for the forehead, where r is the source-detector distance) below, which are taken from Fantini, et. al., “Non-invasive optical monitoring of the newborn piglet brain using continuous wave and frequency domain spectroscopy,” Phys. Med. Biol., 44, 1543-1563 (1999) (“Fantini”), which is incorporated by reference herein.

$$\begin{matrix}DPF = \frac{\sqrt{3 {μ}_{s}^{′}}}{2 \sqrt{{μ}_{a}}} & Eq . \{ 1 \} \\ DPF = \frac{\sqrt{3 {μ}_{s}^{′}}}{2 \sqrt{{μ}_{a}}} \frac{r \sqrt{3 {μ}_{a} {μ}_{s}^{′}}}{r \sqrt{3 {μ}_{a} {μ}_{s}^{′}} + 1} & Eq . \{ 2 \}\end{matrix}$$

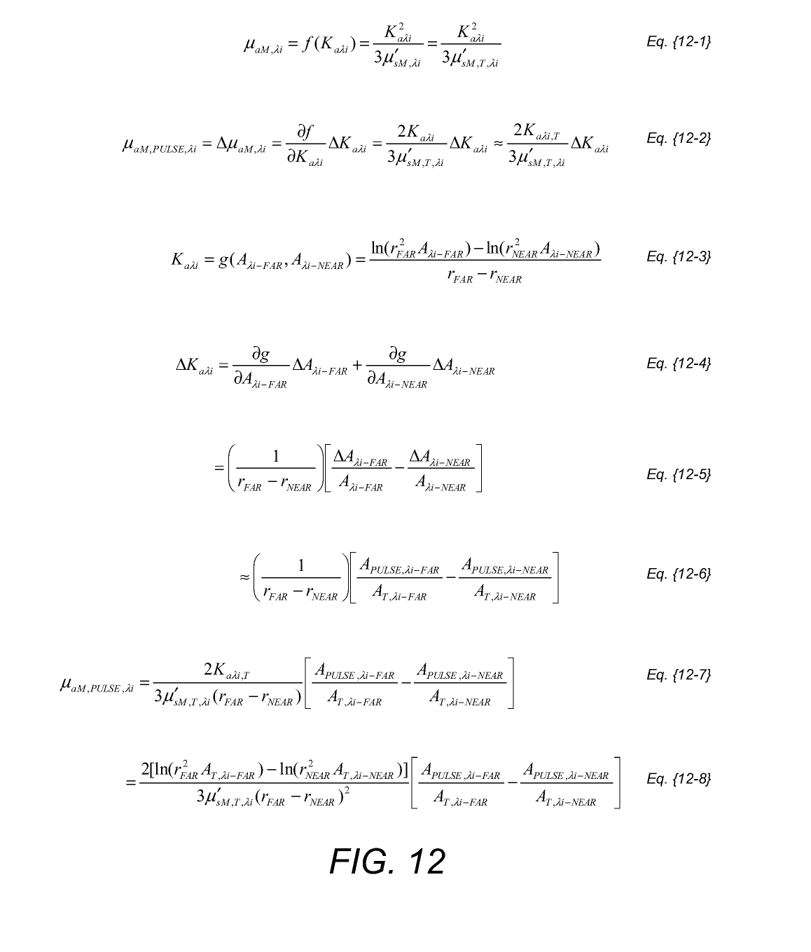

At step 160, the CWS-based measurements are processed in conjunction with the PMS-derived intermediate information to compute a pulsatile component of the absorption property of the biological volume for each of the at least three distinct wavelengths. For preferred embodiments in which the CWS measurements are taken for two or more source-detector pairs at different source-detector distances, the pulsatile component of the absorption property can be computed based on the slope method illustrated in FIGS. 4A-4C in conjunction with the CWS relationship of FIG. 5B at Eq. {5B-1} as differentiated with respect to near and far intensities, which is detailed further with respect to step 812 of FIG. 8 and FIG. 12. For preferred embodiments in which the CWS measurements are taken by only a single source-detector pair, the pulsatile component of the absorption property μ_{a,PULSE} can be computed based on a known relationship between the DPF (as computed at step 158), the source-detector separation distance r, and the measured CWS signal intensities I(max) and I(min) as measured at the pulsatile peaks and valleys thereof, respectively, as expressed in Eq. {3} below, which is adapted from Fantini, supra.

$${μ}_{a , PULSE} = \frac{1}{r DPF} ln ( \frac{I ( max )}{I ( min )} ) \qquad Eq . \{ 3 \}$$

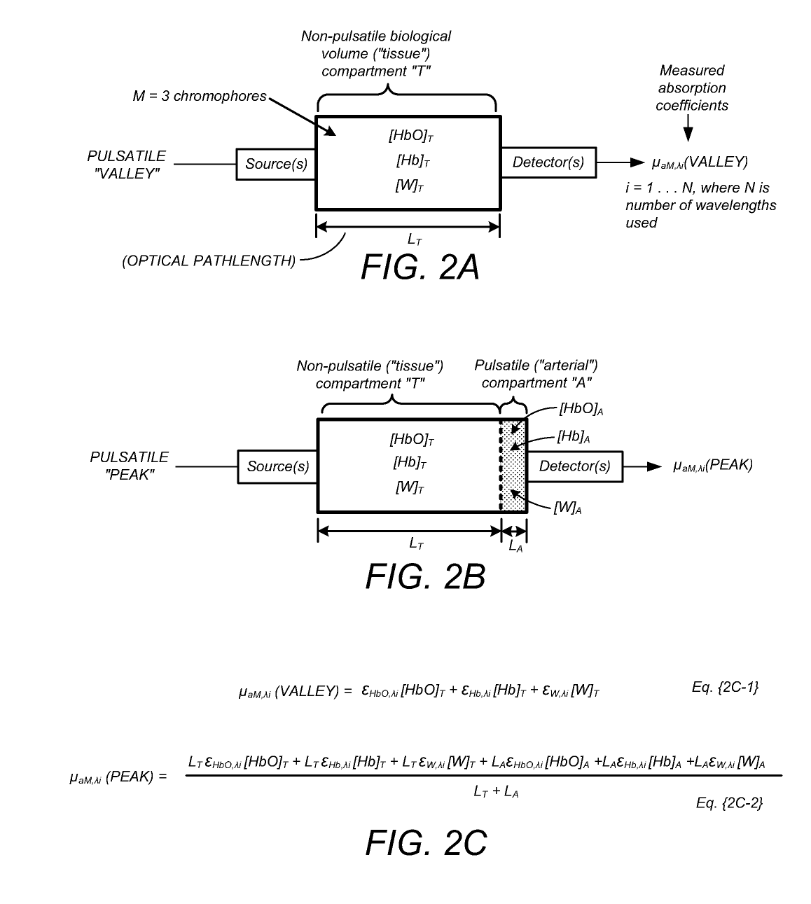

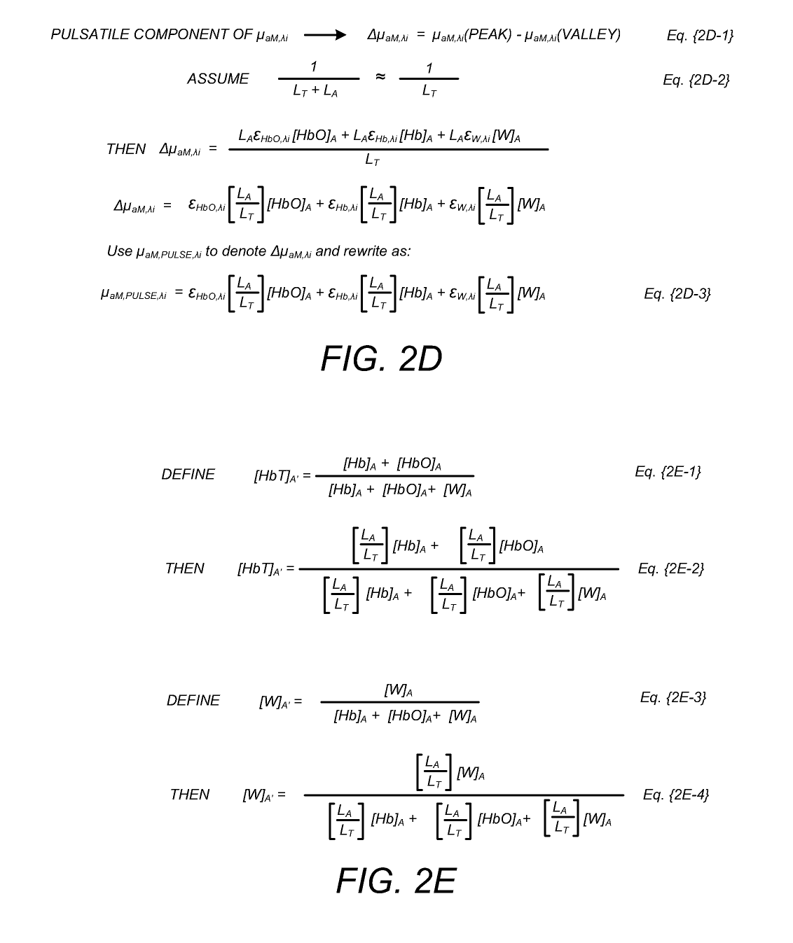

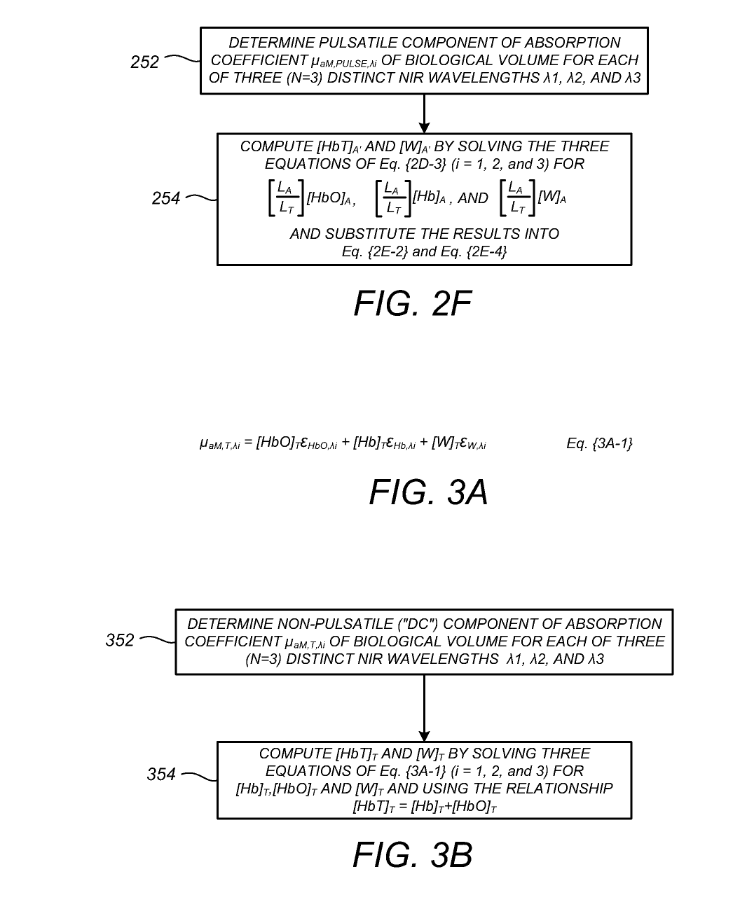

At step 162, at least one metric representative of the at least one chromophore level in the biological volume is computed based on the pulsatile component of the absorption property at the at least three wavelengths. An example of such a computation for a particular example in which at least one metric is an arterial total hemoglobin level metric [HbT]A′ and an arterial water level metric [W]A′ is detailed further hereinbelow with respect to step 254 of FIG. 2F. One example of Finally, at step 164, the at least one metric is displayed on an output display.

FIGS. 2A-2B set forth a compartment model of a biological tissue volume upon which the NIR probe patch 104 (FIG. 1A) or the NIR finger-mounted probe 104′ (FIG. 1D) is mounted. The biological volume consists of a non-pulsatile (“tissue”) compartment “T” and a pulsatile (“arterial”) blood compartment “A.” During a pulsatile “valley” (FIG. 2A), there is only the non-pulsatile tissue compartment “T” between the source and detector having an optical pathlength of L_{T}. During a pulsatile “peak” (FIG. 2B), there is both the non-pulsatile tissue compartment “T” having optical pathlength L_{T} and the pulsatile arterial compartment “A” having an optical pathlength L_{A} between the source and the detector.

FIGS. 2C-2F illustrate mathematical expressions related to the computation of at least one metric representative of a biological constituent level in a biological volume based on computed pulsatile variations of an absorption property thereof at three different wavelengths. More particularly, FIGS. 2C-2F illustrate the model-based mathematical underpinnings for determination of the values of [HbT]_{A′} and [W]_{A′} based upon pulsatile components of the absorption coefficients at three different wavelengths for the biological volume, as measured using the NIR probe patch 104 or the NIR finger-mounted probe 104′. The extinction coefficients (c) of the different components at the different wavelengths are known, predetermined physical constants. In view of the unknown ratio L_{A}/L_{T} it has been found useful to define an arterial hemoglobin concentration metric [HbT]_{A′} as set forth in Eq. {2E-1} and an arterial water concentration metric [W]_{A′} as set forth in Eq. {2E-3}. In an alternative preferred embodiment, the arterial hemoglobin concentration metric [HbT]_{A′} can be defined as set forth in Eq. {2E-1} except with the denominator only being set to [W]_{A}. Although the arterial hemoglobin concentration metric [HbT]_{A′} as set forth in Eq. {2E-1} has been found useful and clinically relevant in its own right, there can be provided in alternative preferred embodiments one or more relatively simple calibration schemes based on experimental data to map the derived value [HbT]_{A′} into the “true” arterial hemoglobin concentration [HbT]_{A} as defined by the relationship [HbT]_{A}=[HbO]_{A}+[Hb]_{A}.

FIGS. 3A-3B illustrate mathematical expressions related to the computation of a biological constituent level in a biological volume based on computed non-pulsatile variations of an absorption property thereof at three distinct wavelengths according to a preferred embodiment. More particularly, FIGS. 3A-3B illustrate the model-based mathematical underpinnings for determination of the values of [HbT]_{T} and [W]_{T} based upon non-pulsatile components of the absorption coefficients at three different wavelengths for the biological volume, as measured using the NIR probe patch 104 or the NIR finger-mounted probe 104′. FIGS. 4A-5C summarize key relationships of the semi-infinite slope method for absorption and effective scattering coefficient computation, which are detailed further in the '218 application, supra.

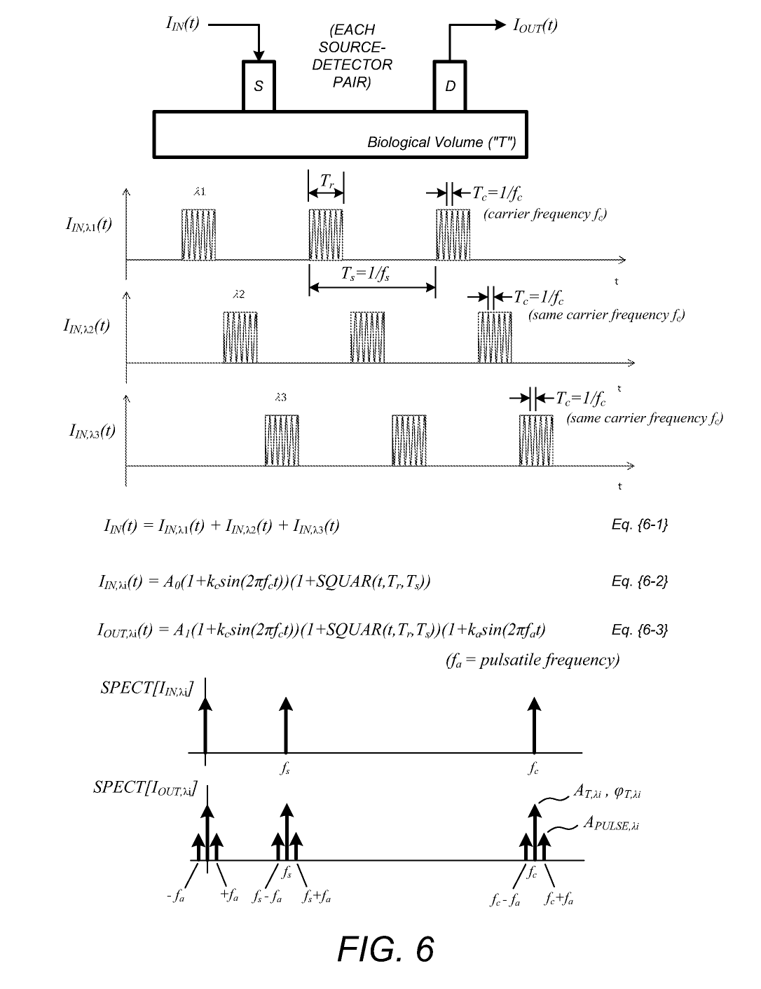

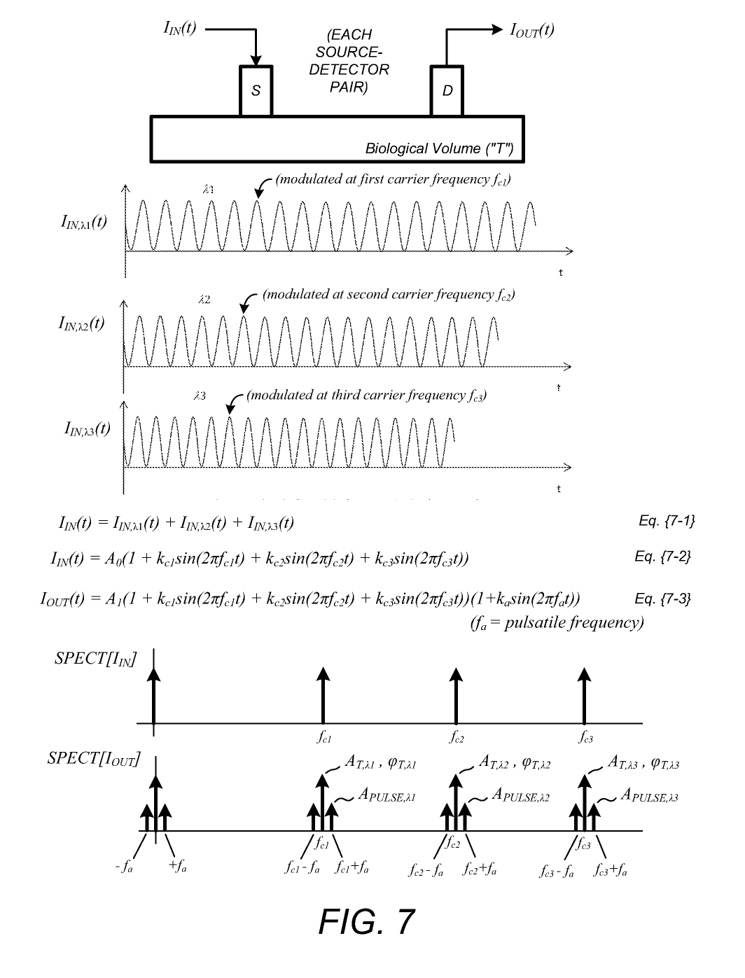

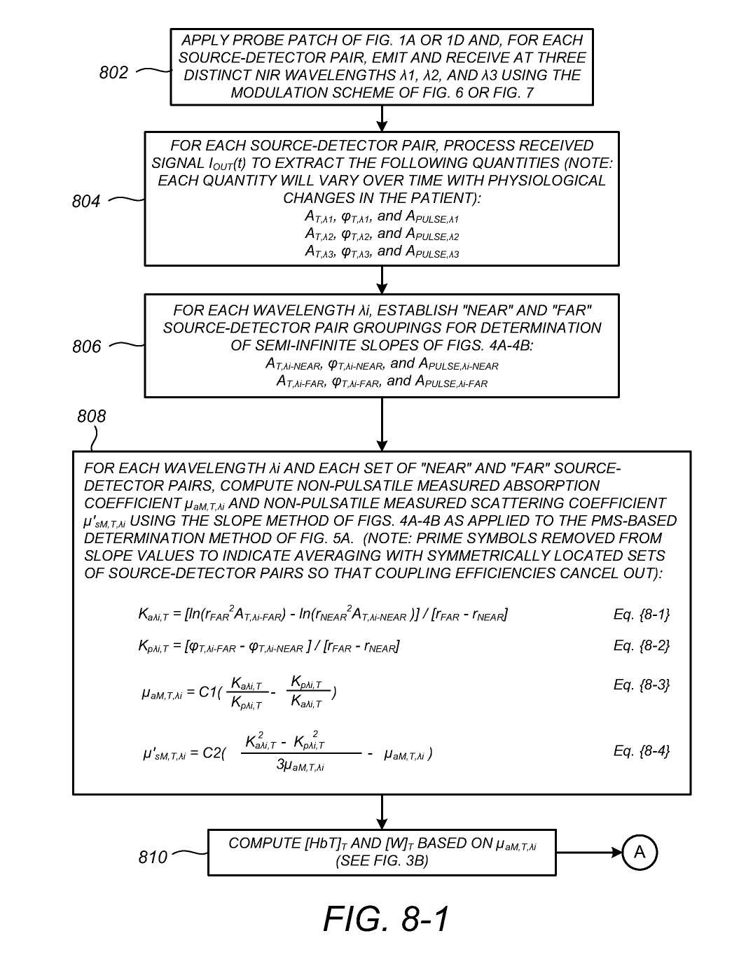

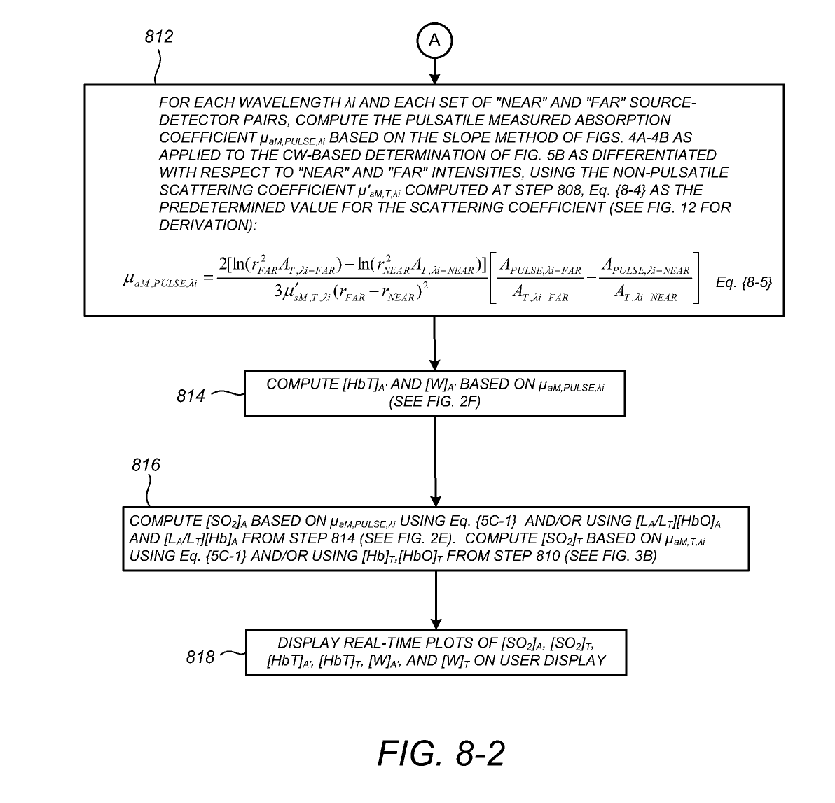

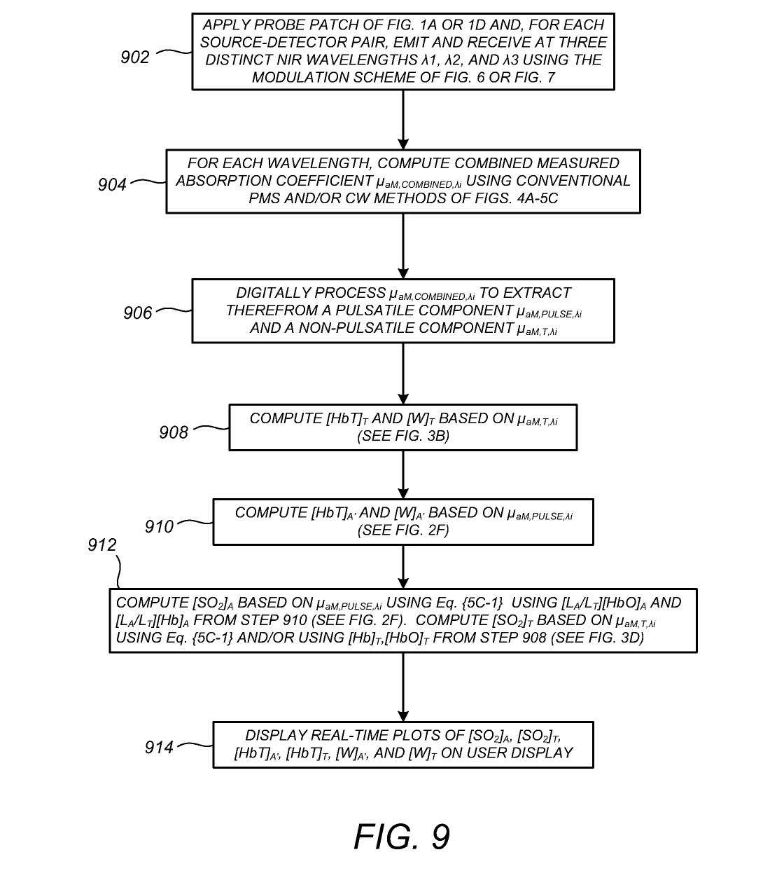

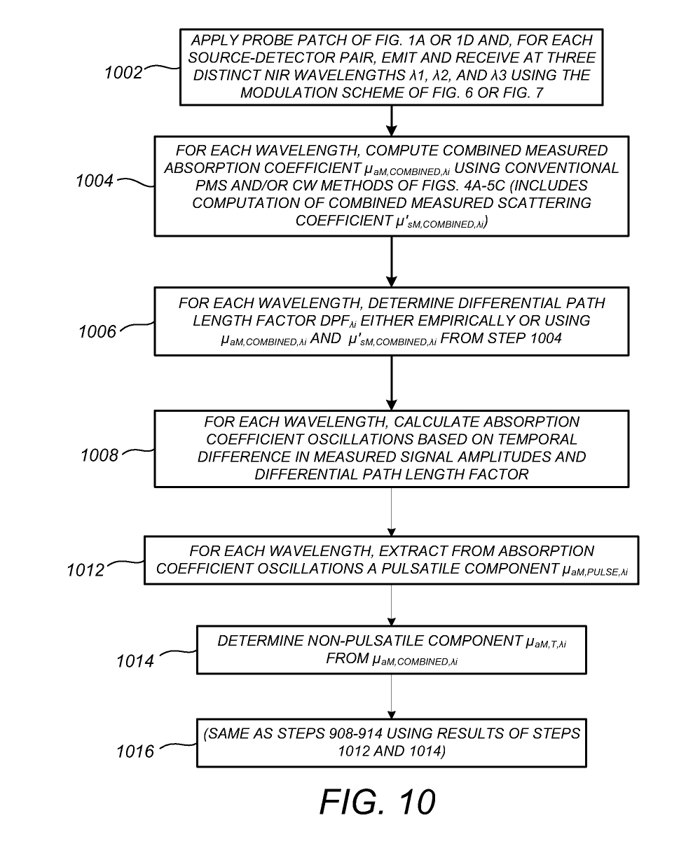

FIG. 6 illustrates a “switching method” for introduction of the optical radiation introduction into the biological volume, wherein a single carrier frequency (e.g., 150 MHz) is used and the source-detector pairs for different wavelengths are operated at distinct intervals. FIG. 7 illustrates a “multiple frequency method” in which different carrier frequencies are used for the different wavelengths, respectively, and in which all wavelengths are emitted and detected at the same time. Generally speaking, either of the schemes of FIG. 6 and FIG. 7 can be used in conjunction with the different computation methods of FIGS. 8, 9, and 10. More generally, any of a variety of schemes for achieving proper timing sequences of the input radiation in view of the various different wavelengths and different modulation schemes are within the scope of the present teachings, including, but not limited to, the schemes set forth in the commonly assigned U.S. Ser. No. 12/832,603, supra. For one preferred embodiment, the waveforms of FIGS. 6-7 are modulated by a much lower-frequency (e.g., 25 kHz) envelope for simultaneously achieving CWS-modulation. For one preferred embodiment, combined PMS and CWS modulation is applied to an optical signal, whereby the same optical signal has a high-frequency PMS modulated signal (e.g., at 150 MHz) contained within a low-frequency (e.g., 25 kHz) CWS-modulated envelope.

By way of further example of the variety of schemes for achieving proper timing sequences of the input radiation that are within the scope of the preferred embodiments, for one preferred embodiment the combined PMS and CWS modulation is applied to an optical signal on a continuous basis, and then the detector equipment alternates between a CWS detection mode and a PMS detection mode at alternating periods of time (PMS detection, then CWS detection, then PMS detection, then CWS detection, etc.). In another preferred embodiment, the optical source transmission scheme can also also alternated between PMS source modulation and CWS source modulation. Thus, for example, there can be a high-frequency PMS modulation of an optical source for a 5-second period, then a low frequency CWS source modulation of the optical source for a 5-second period, then back to PMS, then CWS, then PMS, and so on in alternating 5-second intervals (or, more generally “X” second intervals, it being understood that 5-second intervals are just presented by way of example). The receiving-end detection scheme follows along in a detection mode (CWS or PMS) synchronously with the current mode (CWS or PMS) of the source-end modulation scheme.

FIG. 8 illustrates computation of [HbT]_{A′}, [Hb_{T}]_{T}, [W]_{A′}, [W]_{T}, [SO₂]_{A}, and [SO2]_{T} according to one preferred embodiment, and which generally corresponds in more detail to the general steps set forth in FIG. 1G, supra, wherein a PMS-based computation of the non-pulsatile absorption and effective scattering coefficients is carried out to determine [Hb_{T}]_{T} and [W]_{T}, wherein a CW-based computation of the pulsatile absorption coefficients is carried out to determine [HbT]_{A′} and [W]_{A′}, and wherein the non-pulsatile effective scattering coefficient is used as the effective scattering coefficient in the CW-based computation of the pulsatile measured absorption coefficient. Referring now particularly to step 812, the derivation for computing the pulsatile measured absorption coefficient shown in Eq. {8-5} can be found at FIG. 12. Shown in Eq. {12-1} is the expression for the overall measured absorption coefficient according to the CW-based computation of Eq. {5B}, where the prime symbol is removed from the amplitude slope value K_{aλi} to indicate that averaging has taken place for symmetrically located sets of source-detector pairs so that coupling efficiencies cancel out. Further information on the use of dual sets of near-far source-detector pairs to achieve independence from coupling efficiencies can be found in the '218 application, supra, along with descriptions of alternative methods in which non-symmetric arrangements can be used to yield analogous coupling efficiency-independent results. In Eq. {12-2}, the pulsatile measured absorption coefficient is related with differential changes in the amplitude slope value, which in turn is related with differential changes in the measured near and far intensity amplitudes as developed in Eqs. {12-3}-{12-6}. Finally, by the substitutions shown in Eqs. {12-7}-{12-8}, the pulsatile measured absorption coefficient can be expressed in terms of the measured pulsatile and non-pulsatile “near” and “far” intensity amplitudes and the non-pulsatile effective scattering coefficient, as shown in Eq. {12-8}, which in turn is copied as Eq. {8-5} in FIG. 8.

FIG. 9 illustrates computation of [HbT]_{A′}, [HbT]_{T}, [W]_{A′}, [W]_{T}, [SO₂]_{A}, and [SO₂]_{T} according to another preferred embodiment in which the combined (i.e., pulsatile and non-pulsatile combined) measured absorption coefficient is computed as a whole, and then the pulsatile and non-pulsatile components thereof are extracted. The method of FIG. 9 is believed to be somewhat disadvantageous from a dynamic range perspective, in view of the fact that the arterial pulsations in the combined measured absorption coefficient will be relatively small compared to its non-pulsatile component.

FIG. 10 illustrates computation of [HbT]_{A′}, [HbT]_{T}, [W]_{A′}, [W]_{T}, [SO₂]_{A}, and [SO₂]_{T} according to another preferred embodiment in which a temporal and DPF-based method is used to compute the pulsatile component of the absorption coefficient, wherein an effective scattering coefficient computed from a PMS-based computation of the combined absorption coefficient is used as a basis for computing the DPF (differential path length factor). As mentioned above with respect to FIGS. 1C and 1F, different PMS-based methods other than the slope method can be used to compute the DPF and the relevant absorption and reduced scattering coefficients when only a single source-detector pair is present.

FIG. 11 illustrates conceptually how the pulse signal P(t) can be derived from a single detector signal intensity by controlled DC component removal and pulsatile component amplification. The switching frequency and carrier frequency, including multi-frequency transmission, is removed. A DC elimination unit subtracts a DC signal provided by a DC processing unit from the demodulated signal, to generate a pulsatile-only signal, which is then amplified. The amplified pulsatile signal is then digitized for processing, such as for use in determining A_{PULSE, λi} (see FIGS. 6-8).

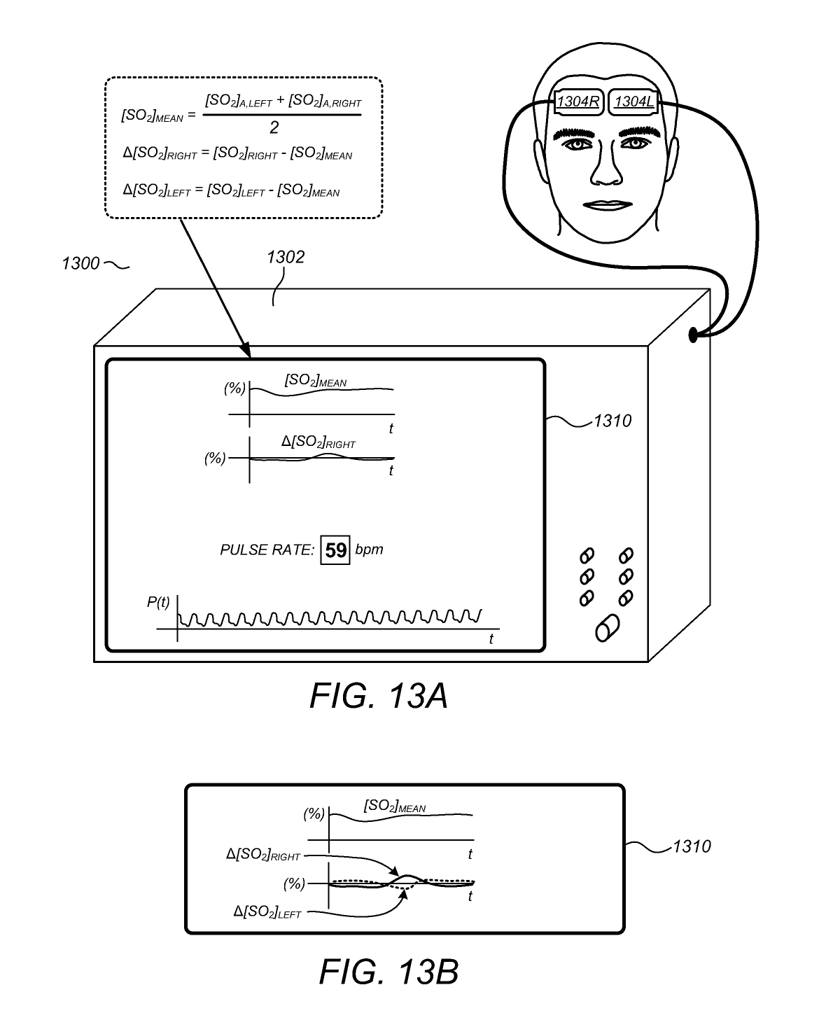

FIG. 13 illustrates a bilateral cerebral pulse oximetry system 1300 according to a preferred embodiment in which left-side and right-side SO₂ readings are computed, and then the clinical results are effectively communicated to the medical professional in a manner that does not require a simultaneous dual-trace display of left-side and right-side SO₂ readings. In particular, the display 1310 in FIG. 13A shows a trace of a mean SO₂ reading (average of the left and right sides), and therebelow shows a trace of the difference between the right side SO₂ reading and the mean reading. The preferred embodiment of FIG. 13B adds a trace of the difference between the left side SO₂ reading and the mean reading. Similar trace displays can be provided for other left-right localized readings such as [HbT]ₓ, [HbT]_{T}, [W]_{A′}, [W]_{T}, and so forth.

For one preferred embodiment, PMS-based modulation and processing is used as a modifying adjunct for a pre-existing non-PMS-based monitoring system for supplying one or more key intermediate quantities pertaining thereto. One example of a key intermediate quantity is a differential pathlength factor (DPF), although there can be a variety of others without departing from the scope of the present teachings. The key intermediate quantity is a factor, computed feature, or relationship that is normally used by the pre-existing non-PMS-based monitoring system as part of its computations, and which is capable of being provided by a PMS-based system. As in the particular example of the DPF, non-PMS-based systems often resort to assumptions, complex calibrations schemes, or other workarounds to derive a suitable value for that quantity, whereas PMS-based systems can directly measure or otherwise provide a better, more reliable, and/or higher-quality version of that quantity.

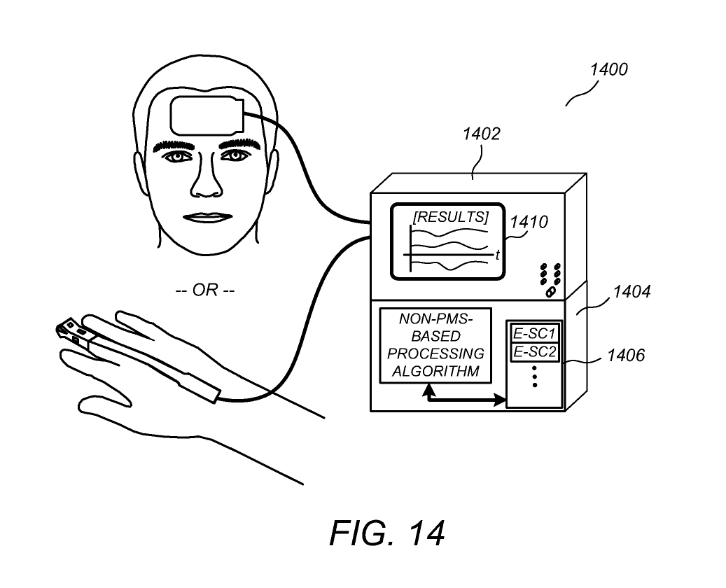

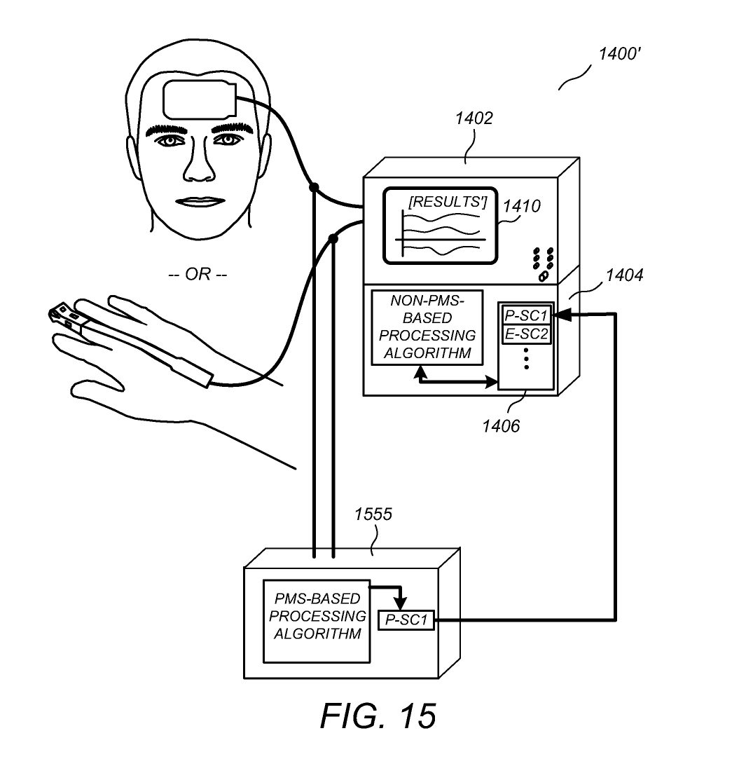

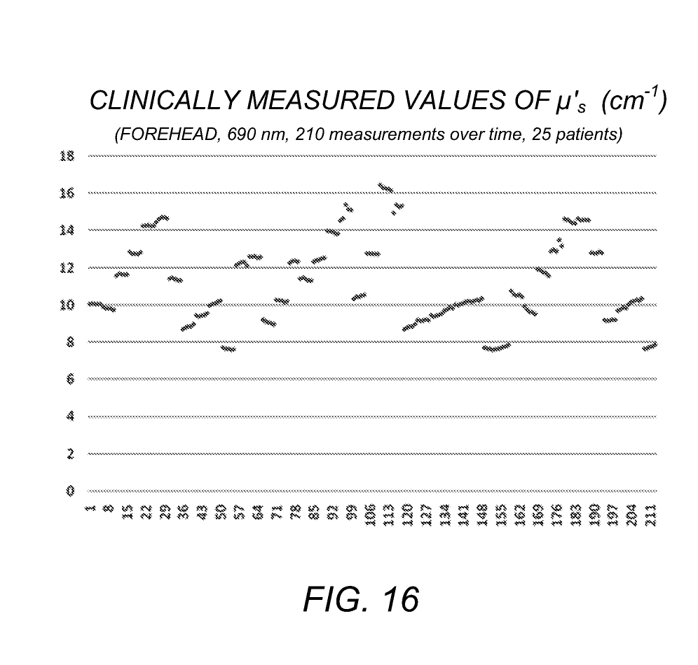

FIGS. 14-15 illustrate an advantageous modification of a non-PMS-based NIRS monitoring system with certain aspects of a PMS-based monitoring system according to a preferred embodiment. FIG. 14 illustrates a pre-existing non-PMS-based monitoring system 1400 that, while highly perfected in several respects, may still suffer from inaccuracy in that certain intermediate quantities (generally spectrophotometric characteristics) are estimated, and wherein the accuracy could be increased if those intermediate quantities were provided by a PMS-based system. For example, the non-PMS system 1400 can be a CWS system that depends on a pre-existing estimate of a scattering property, DPF, or other scatter-related property of the biological volume. The non-PMS-based monitoring system 1400 comprises a console 1402 including a processing unit 1404 that executes a non-PMS-based algorithm. As part of that algorithm, the processing unit 1404 includes a memory 1406 where there is stored one or more estimated intermediate quantities E-SC1, E-SC2, etc., where E-SC stands for an estimated spectrophotometric characteristic. One example of an E-SC is a DPF (differential pathlength factor). Other examples of E-SC can include, without limitation, estimated absorption coefficient(s), estimated reduced scattering coefficient(s), estimated optical pathlength(s), and estimated phase measurement(s) as may be needed

FIG. 15 illustrates a hybrid PMS/non-PMS NIRS monitoring system 1400′ according to a preferred embodiment, comprising generally the non-PMS-based monitoring system 1400 but into which is integrated a second processing unit 1555 that implements a PMS-based processing algorithm. The hybrid PMS/non-PMS NIRS monitoring system 1400′ further includes hardware upgrades to the probe patch(es) and/or finger-mounted probe(s), such as the inclusion of laser diodes and driving circuitry as needed for high PMS-based modulation rates (e.g., 150 MHz and higher), such upgrades being achievable by a person skilled in the art in view of the present disclosure and not being detailed in FIG. 15. According to a preferred embodiment, the second processing unit 1555 computes an actual version P-SC1 of the estimated spectrophotometric characteristic E-SC1 (such as a DPF, for example), and then that value is inserted into memory 1406 and used by the non-PMS-based processing algorithm to achieve results that are displayed on the display 1410. Advantageously, the displayed results computed using the more perfect value P-SC1 in hybrid system 1400′ of FIG. 15 are improved over those computed using the less perfect value E-SC1 in system 1400 of FIG. 14. Examples of pre-existing non-PMS-based monitoring system 1400 that may benefit from the preferred embodiment of FIG. 15 include, but are not limited to, devices based on Masimo Rainbow SET® Measurement technology, and devices based on Somanetics INVOS® technology, each of which is non-PMS-based.

Described hereinbelow is an alternative to the above-described hybrid PMS-CWS (and, more generally hybrid PMS-non-PMS) methods above for computing a blood total hemoglobin concentration [HbT]_{A}. The above-described methods are generally founded upon a medical premise that arterial blood vessels in the biological volume under surveillance will pulsate with the heartbeat of the patient, expanding to a “peak” volume and contracting again to a “valley” volume with each heartbeat. Therefore, any differential variations in the NIRS measurement signals occurring at the pulsatile frequency can be directly associated with the differential amount of blood (specifically, arterial blood, since the venous blood vessels do not pulsate) present in the biological volume under surveillance. As disclosed above, extraction of the pulsatile components of the NIRS measurement signals (also termed the “AC” components) from the non-pulsatile components of the NIRS measurement signals (also termed the “DC” components) provides an ability to specifically identify the blood total hemoglobin concentration [HbT]_{A} in the biological volume under surveillance. Notably, the blood total hemoglobin concentration [HbT]_{A} is substantially different than the overall total hemoglobin concentration [HbT]_{T} in the biological volume under surveillance, because the biological volume under surveillance will always include many other biological items in addition to blood, such as intracellular fluid, interstitial fluid, bone, and so forth. Thus, the overall hemoglobin concentration [HbT]_{T} is not specific to the blood itself, and represents a more generic, less targeted measurement than the blood total hemoglobin concentration [HbT]_{A}.

Although there certainly is a sound basis for extraction of the pulsatile (“AC”) components of the NIRS measurement signals to compute blood total hemoglobin concentration [HbT]_{A}, as set forth above, practical issues can arise in extracting the relatively weak pulsatile (“AC”) components of the NIRS measurements in a manner sufficiently reliable to achieve good clinical results for a variety of different body parts, monitoring conditions, and patient conditions. It may be desirable to provide an alternative and/or adjunctive method to monitor blood total hemoglobin concentration [HbT]_{A} in which extraction of pulsatile (“AC”) components of the NIRS measurements is not required.

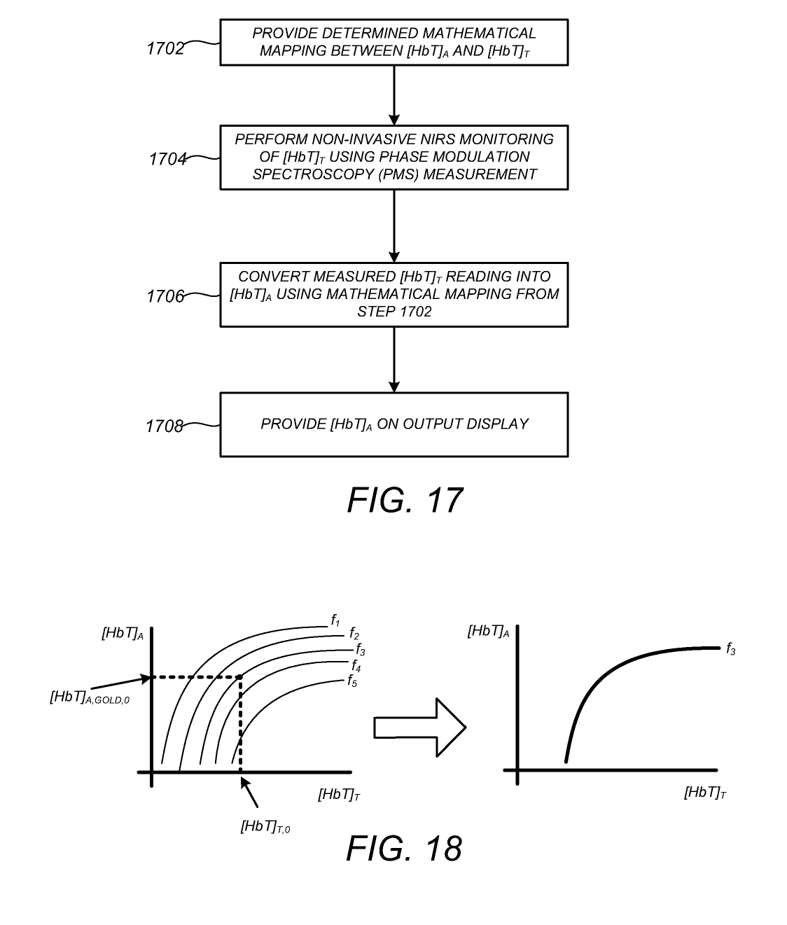

FIG. 17 illustrate continuous, real-time, non-invasive NIRS monitoring of blood total hemoglobin concentration [HbT]_{A} monitoring according to a preferred embodiment, in which extraction of pulsatile signal components is not required. At step 1702, a mathematical mapping is determined between measured tissue total hemoglobin concentration [HbT]_{T} and blood total hemoglobin concentration [HbT]_{A}. It should be appreciated that although the subscript “A” can be seen in the term [HbT]_{A}, the concentration [HbT]_{A} applies to both arterial and venous blood alike, since venous and arterial blood have the same total hemoglobin concentrations. At step 1704, the tissue total hemoglobin concentration [HbT]_{T} is continuously and non-invasively measured using phase modulation spectroscopy (PMS) NIRS methods. At step 1706, the measured tissue total hemoglobin concentration [HbT]_{T} is converted into a blood total hemoglobin concentration [HbT]_{A} using the predetermined mathematical mapping from step 1702. Finally, at step 1708, the blood total hemoglobin concentration [HbT]_{A} is provided on a continuous readout display. In one preferred embodiment, the blood total hemoglobin concentration [HbT]_{A} is provided as an “absolute” metric on the readout display, in graphical and/or numerical format, with units of grams per deciliter (or equivalent concentration units). In another preferred embodiment, there is provided a “relative” blood total hemoglobin concentration readout, which is provided as a trend graph and/or in numerical percentage format, relative to a clinically convenient baseline value, such as a value established at the beginning of a monitoring session. Although an “absolute” blood total hemoglobin concentration readout is of course preferable, the latter “relative” blood total hemoglobin concentration readout can still provide useful trend data, and could provide a fallback in the event that inter-patient variation issues, governmental clearance issues, or other real-world factors make the provision of “absolute” readings impracticable on a per-patient basis, a per-model basis, or on some other basis.

With reference to step 1704 of FIG. 1, according to a preferred embodiment, the biological volume “T” to be monitored is considered as a single compartment fully and homogeneously occupied by biological material containing a group of “N” different chromophores, N≧4. The group of “N” chromophores includes a first chromophore that is oxygenated hemoglobin having a concentration [HbO]_{T} and a set of known wavelength-specific extinction coefficients ε_{HbO,λi}. The group of “N” chromophores further includes a second chromophore that is deoxygenated hemoglobin having a concentration [Hb]_{T} and a set of known wavelength-specific extinction coefficients ε_{Hb,λi}. The group of “N” chromophores further includes “N−2” additional chromophores Xₙ, n=3 . . . N, each having a concentration [Xₙ] and each having its own set of known wavelength-specific extinction coefficients ε_{Xn,λi}. Examples of the additional “N−2” chromophores can include water, glucose, albumin, lipids, fibrous cellular tissue, CO, methemoglobin, and so forth according to the model to be used. Using appropriate probe patches and phase modulation spectroscopy (PMS) NIRS hardware as described elsewhere in the incorporated commonly assigned patent applications supra, the absorption coefficient λ_{a,meas,λi} of the biological volume is measured for each of “M” different NIRS wavelengths λᵢ, where i=1 . . . M. The number of NIRS wavelengths M is at least as great as the number of chromophores “N” in the model of the biological volume. For the particular example of M=N=4, an exemplary set of wavelengths can be λ₁=680 nm, λ₂=730 nm, λ₃=780 nm, and λ₄=830 nm. By solving the set of equations set forth below for the particular example of M=N=4 and the above-referenced wavelengths (which is readily extendible for other values of M and N and other wavelengths) the values for [Hb]_{T} and [HbO]_{T} can be determined:

μ_{a,meas,680}=ε_{HbO,680}[HbO]_{T}+ε_{Hb,680}[Hb]_{T}+ε_{X3,680}[X₃]_{T}+ε_{X4,680}[X₄]_{T}

μ_{a,meas,730}=ε_{HbO,730}[HbO]_{T}+ε_{Hb,730}[Hb]_{T}+ε_{X3,730}[X₃]_{T}+ε_{X4,730}[X₄]_{T}

μ_{a,meas,780}=ε_{HbO,780}[HbO]_{T}+ε_{Hb,780}[Hb]_{T}+ε_{X3,780}[X₃]_{T}+ε_{X4,780}[X₄]_{T}

μ_{a,meas,830}=ε_{HbO,830}[HbO]_{T}+ε_{Hb,830}[Hb]_{T}+ε_{X3,830}[X₃]_{T}+ε_{X4,830}[X₄]_{T} {Eq. 4}:

Then, using the known relationship [HbT]_{T}=[Hb]_{T}+[HbO]_{T}, the value for [HbT]_{T} can be determined on an ongoing basis using the acquired PMS NIRS measurements. It is most advantageous to use PMS NIRS measurements over other NIRS techniques such as continuous wave (CWS) techniques, because the PMS NIRS measurement methods will not require non-measured estimations of scattering coefficients or path length factors, and therefore the measured values for the absorption coefficients at the left side of Eq. {4} will be more reliable and precise.

With reference to step 102 of FIG. 1, the mathematical mapping between measured tissue total hemoglobin concentration [HbT]_{T} and blood total hemoglobin concentration [HbT]_{A} can be achieved by creating a model mathematical relationship between [HbT]_{T} and [HbT]_{A} having one or more model parameters, and then determining the model parameters based on empirical data acquired using a population of human test subjects, test phantoms, and/or test animals to which is applied the non-invasive PMS NIRS measurement system and a “gold” reference measurement (or other known method of determination) for actual [HbT]_{A} values. Different model structures and/or parameters can be employed for different body parts that are being non-invasively monitored (e.g. a first model/parameter set for the forehead, a second model/parameter set for the neck, a third model/parameter set for the forearm, and so forth).

For one preferred embodiment, the mathematical relationship for step 1702 can be universal and predetermined, in which case the entire monitoring process can be non-invasive. Optionally, the mathematical relationship can be provided as a lookup table, wherein the lookup table can be pre-calibrated based on a variety of criteria including (a) probe location on the body, (b) patient age, (c) patient gender, (d) patient temperature, and so forth.

According to another preferred embodiment that is particularly advantageous for clinical hospital settings such as a post-surgical and/or intensive care unit environment, the mathematical relationship for step 1702 can be based on a combination of predetermined empirical relationships/lookup tables together with a single invasive measurement that specifically calibrates the system to the particular patient being monitored. This is shown conceptually in FIG. 18, which shows a plurality of different possible pre-established, mappings fⱼ between [HbT]_{T} and [HbT]_{A}. At the beginning of the monitoring procedure, a single invasive blood sample can be drawn from the patient and chemically analyzed (or otherwise subjected to “gold standard” measurement) to determine a true initial reading [HbT]_{A,GOLD,0}. At the same time, the non-invasive PMS NIRS system is applied to the relevant location of the patient to obtain a non-invasive initial reading [HbT]_{T,0}. Then, as graphically illustrated in FIG. 18, the readings [HbT]_{A,GOLD,0} and [HbT]_{T,0} can be used to select one of a predetermined number of possible functional mappings between [HbT]_{A} and [HbT]_{T}, and that selected mapping will be used thereafter in that monitoring session for that patient. Thus, whereas conventional post-surgical and/or intensive care unit environments would require periodic physical blood draws from the patient (for example, one blood draw every 4 hours) and chemically analysis of those samples to ensure that the patient has appropriate [HbT]_{A} levels (for example, to ensure that there is no internal bleeding in the patient), a method according to the currently described preferred embodiment only requires a single physical blood draw and chemical analysis at the outset of the monitoring session, and thereafter the non-invasive method of FIGS. 17-18 can be used as reliable determinants of [HbT]_{A} levels.

Whereas many alterations and modifications of the present invention will no doubt become apparent to a person of ordinary skill in the art after having read the foregoing description, it is to be understood that the particular embodiments shown and described by way of illustration are in no way intended to be considered limiting. By way of example, while the PMS measurement methodologies associated with one or more preferred embodiments are described above as having two or more source-detector pairs at different distances for accommodating the so-called “slope method” in the computation of the non-pulsatile absorption property and the scattering property of the biological volume, it is not outside the scope of the present teachings for only a single source-detector pair, or fewer pairs than needed for the slope method, to be used. In such case, known or hereinafter developed PMS measurement methodologies based on the use of a single source-detector pair, or fewer pairs than needed for the slope method, can be used in the determination of the non-pulsatile absorption property and the scattering property (or PMS-derived intermediate information representative of the scattering property). Therefore, reference to the details of the embodiments are not intended to limit their scope, which is limited only by the scope of the claims set forth below.

What is claimed is:

1. A method for near-infrared spectrophotometric (NIRS) monitoring of at least one chromophore level in a biological volume of a patient, comprising:
determining a non-pulsatile component of an absorption property of the biological volume for each of at least three distinct wavelengths of near-infrared radiation using a phase modulation spectrophotometry (PMS) based measurement method, said PMS-based measurement method being characterized by a relatively high modulation rate and being further characterized in that both amplitude and phase information detected at the relatively high modulation rate are processed to compute said non-pulsatile component of the absorption property;
processing the measured amplitude and the measured phase information associated with said PMS-based determination of said non-pulsatile component of the absorption property to compute PMS-derived intermediate information that is at least partially representative of a scattering characteristic of the biological volume;
determining a pulsatile component of the absorption property of the biological volume for each of said at least three distinct wavelengths using a continuous wave spectrophotometry (CWS) based measurement method, said CWS-based measurement method being characterized by a relatively low modulation rate, wherein said determining the pulsatile component of the absorption property comprises processing amplitude information detected at the relatively low modulation rate in conjunction with said PMS-derived intermediate information to compute said pulsatile component of the absorption property;
computing at least one metric representative of the at least one chromophore level in the biological volume based on said pulsatile component of the absorption property at said at least three wavelengths; and
displaying said at least one metric on an output display.

2. The method of claim 1, wherein said PMS-derived intermediate information comprises a scattering property for each of said at least three wavelengths.

3. The method of claim 1, wherein said PMS-derived intermediate information comprises a differential pathlength factor (DPF) for each of said at least three wavelengths.

4. The method of claim 1, wherein said relatively high modulation rate associated with said PMS-based measurement method is greater than about 100 MHz, and wherein said relatively low modulation rate associated with said CWS-based measurement method is less than about 1 MHz.

5. The method of claim 1, wherein said PMS-based measurement of said non-pulsatile component of the absorption property is carried out using a same set of source-detector pairs as are used in carrying out said CWS-based measurement of said pulsatile component of the absorption property.

6. The method of claim 1, wherein said PMS-based measurement of said non-pulsatile component of the absorption property is carried out using a different set of source-detector pairs as are used in carrying out said CWS-based measurement of said pulsatile component of the absorption property.

7. The method of claim 1, wherein said PMS-based measurement of said non-pulsatile component of the absorption property is carried out using a plurality of source-detector pairs at different source-detector spacings, and wherein said CWS-based measurement of said pulsatile component of the absorption property is carried out using a single one of said source-detector pairs.

8. The method of claim 1, wherein said at least one metric includes an arterial total hemoglobin metric and an arterial water level metric.

9. The method of claim 8, wherein said arterial total hemoglobin metric corresponds to a ratio of an arterial total hemoglobin concentration for the biological volume to a sum of the arterial total hemoglobin concentration and an arterial water concentration for the biological volume.

10. The method of claim 8, further comprising:
processing the measured amplitude and the measured phase information associated with said PMS-based determination of said non-pulsatile component of the absorption property to compute a tissue total hemoglobin concentration and a tissue water concentration for the biological volume; and
displaying said tissue total hemoglobin concentration and said tissue water concentration on the output display in conjunction with said arterial total hemoglobin metric and said arterial water level metric.

11. The method of claim 10, further comprising:
processing the measured amplitude and the measured phase information associated with said PMS-based determination of said non-pulsatile component of the absorption property to compute an oxygen saturation metric for the biological volume; and
displaying said oxygen saturation metric on the output display.

12. An apparatus for non-invasive near-infrared spectrophotometric (NIRS) monitoring of at least one chromophore level in a biological volume of a patient, comprising:
a probe assembly including a plurality of source-detector pairs configured to introduce near-infrared radiation into the biological volume and receive near-infrared radiation from the biological volume;
a processing and control device coupled to said plurality of source-detector pairs of said probe assembly, the processing and control device being configured to operate at least one of said source-detector pairs in a phase modulation spectrophotometry (PMS) mode, said PMS mode being characterized by a relatively high modulation rate and being further characterized in that both amplitude and phase information are detected and processed to determine an absorption property, the processing and control device being further configured to operate at least one of said source-detector pairs in a continuous wave spectrophotometry (CWS) mode, said CWS mode being characterized by a relatively low modulation rate and being further characterized in that amplitude information is detected and processed to determine the absorption property without regard to phase information; and
an output display coupled to said processing and control device;
wherein said processing and control device is programmed and configured in conjunction with said plurality of source-detector pairs and said output display to carry out the steps of:
determining a non-pulsatile component of an absorption property of the biological volume for each of at least three distinct wavelengths based on measurements acquired in said PMS mode;
processing said measurements acquired in said PMS mode to compute PMS-derived intermediate information that is at least partially representative of a scattering characteristic of the biological volume;
determining a pulsatile component of the absorption property of the biological volume for each of said at least three distinct wavelengths based on measurements acquired in said CWS mode, including processing said CWS-mode measurements in conjunction with said PMS-derived intermediate information to compute said pulsatile component of the absorption property;
computing at least one metric representative of the at least one chromophore level in the biological volume based on said pulsatile component of the absorption property at said at least three wavelengths; and
displaying said at least one metric on said output display.

13. The apparatus of claim 12, wherein said PMS-derived intermediate information comprises one of (i) a scattering property for each of said at least three wavelengths, and (ii) a differential pathlength factor (DPF) for each of said at least three wavelengths.

14. The apparatus of claim 12, wherein said relatively high modulation rate associated with said PMS mode is greater than about 100 MHz, and wherein said relatively low modulation rate associated with said CWS mode is less than about 1 MHz.

15. The apparatus of claim 12, wherein a first subset of source-detector pairs on said probe assembly is operable in said CWS mode and a second subset of source-detector pairs on said probe assembly is operable in said PMS mode.

16. The apparatus of claim 15, wherein each of said first subset of source-detector pairs has an optical source in common with a respective one of said second subset of source-detector pairs, said optical source being simultaneously modulated at said relatively high frequency associated with said PMS mode and said relatively low frequency associated with said CWS mode, and wherein each of said first subset of source-detector pairs has an optical detector that is distinct from that of the respective one of the second subset of source-detector pairs.

17. The apparatus of claim 12, wherein said at least one metric includes an arterial total hemoglobin metric corresponding to a ratio of an arterial total hemoglobin concentration for the biological volume to a sum of the arterial total hemoglobin concentration and an arterial water concentration for the biological volume.

18. A method for providing an improved apparatus for near-infrared spectrophotometric (NIRS) monitoring of at least one chromophore level in a biological volume of a patient, comprising:
acquiring a pre-existing NIRS monitoring apparatus including a probe assembly, a processing and control device, and an output display, the pre-existing NIRS monitoring apparatus being operable in a pre-existing continuous wave spectrophotometry (CWS) mode characterized in that (i) a relatively low modulation rate is used, (ii) amplitude information is detected and processed according to a pre-existing algorithm to determine an absorption property without regard to phase information, and (iii) the pre-existing algorithm incorporates a pre-existing estimate of a scatter-related characteristic of the biological volume in the determination of a pulsatile absorption property, the pre-existing NIRS monitoring apparatus computing the at least one chromophore level based on the pulsatile absorption property and displaying the at least one chromophore level on the output display;
modifying said probe assembly and said processing and control device of the pre-existing NIRS monitoring apparatus to be operable in a phase modulation spectrophotometry (PMS) mode in addition to said pre-existing CWS mode, said PMS mode being characterized by a relatively high modulation rate and being further characterized in that both amplitude and phase information are detected; and
further modifying said processing and control device to be operable to:
compute an actual version of said scatter-related characteristic for the biological volume based on measurements acquired in said PMS mode; and
incorporate said actual version of said scatter-related characteristic in place of said pre-existing estimate thereof in said pre-existing algorithm that determines the pulsatile absorption property;
whereby the modified version of the pre-existing NIRS monitoring apparatus provides improved monitoring of the at least one chromophore level by virtue of incorporating an actual, patient-specific, updated version of said scatter-related characteristic in place of the pre-existing estimate thereof in computing the at least one chromophore level.

19. The method of claim 18, wherein said pre-existing estimate of the scatter-related characteristic used by the pre-existing algorithm is one of (i) an pre-estimated scattering property, (ii) a pre-estimated differential pathlength factor (DPF), and (iii) a quantity that is computed from one of the pre-estimated scattering property and the pre-estimated DPF.

20. The method of claim 18, wherein said relatively high modulation rate associated with said PMS mode is greater than about 100 MHz, and wherein said relatively low modulation rate associated with said CWS mode is less than about 1 MHz.
